(19) United States
(12) Patent Application Publication
(10) Pub. No.: US 2005/0250113 A1
(43) Pub. Date: Nov. 10, 2005

(54) Mammalian magnesium/manganese sensing G protein coupled receptor

(75) Inventors: Charles S. Zuker, San Diego, CA (US); Isolda Erlenbach, (US); Mark A. Hoon, Kensington, MD (US); Nicholas J.P. Ryba, Bethesda, MD (US); Yifeng Zhang, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: The Regents of the University of California; The Government of the USA as represented by the Secretary of the Dept of Health and Human Services

(21) Appl. No.: 10/841,129

(22) Filed: May 7, 2004

(51) Int. Cl. C12Q 1/68; G01N 33/53; G01N 33/567

(52) U.S. Cl. 435/6; 435/7.2

(57) ABSTRACT

The present invention provides isolated nucleic acid and amino acid sequences of magnesium and manganese-sensing G-protein coupled receptor polypeptides from the C family of G-protein coupled receptors, antibodies to such receptors, methods of detecting such nucleic acids and receptors, and methods of screening for modulators of magnesium and manganese metabolism.

### CROSS-REFERENCES TO RELATED APPLICATIONS

Not applicable.

### STATEMENT AS TO RIGHTS TO INVENTIONS MADE UNDER

### FEDERALLY SPONSORED RESEARCH AND DEVELOPMENT

Not applicable.

### FIELD OF THE INVENTION

The present invention provides isolated nucleic acid and amino acid sequences of magnesium and manganese-sensing G-protein coupled receptor polypeptides from the C family of G-protein coupled receptors, antibodies to such receptors, methods of detecting such nucleic acids and receptors, and methods of screening for modulators of magnesium and manganese sensing and metabolism.

### BACKGROUND OF THE INVENTION

Magnesium is an essential ion and one of the most important micronutrients required for many biochemical and biological processes, ranging from bone and immune system function to modulation of the nervous system and endocrine pathways. The mechanism(s) by which magnesium levels are “sensed” have been intensely studied, yet the critical biological “sensors” have not been identified (see, e.g., Schlingmann et al. Nature Genetics 31:166-170 (2002); Vetter and Lohse, Curr Opin Nephrol Hypertens 1:403-410 (2002; Tam et. al. Europ. Jour. of Chemical Nutrition 57:1193-1197 (2003; and Hofer and Brown, Nature Reviews 4:530-538 (2003)).

The control of magnesium levels is tightly regulated, and its dysfunction leads to sever hypo- and hypermagnesemia disorders, including cardiac arrhythmias, disturbances in conduction, ventricular fibrillation, cardiac arrest, anorexia, nausea, vomiting, lethargy, paresthesia, muscular cramps, irritability, and mental confusion. Hypermagnesemia syndromes can be seen in infants born to pre-eclamptic and eclamptic mothers and subjects with adrenal insufficiency, renal disease, and rhabdomyolysis. Hypomagnesemia syndromes can be seen subjects with familial malabsorption, malnutrition, total parenteral nutrition, malabsorption syndromes (e.g., enteric fistulas, Whipple's disease, intestinal lymphoma, biliary diversion, giardiasis, and short bowel syndrome), specific absorptive defects, prolonged diarrhea, pancreatitis, endocrine disorders (e.g., hyperparathyroidism, hypoparathyroidism, hyperthyroidism, primary hyperaldosteronism, Bartter's syndrome, Gitelman's syndrome, and diabetic and alcoholic ketoacidosis), cellular uptake or redistribution (e.g., administration of epinephrine, massive blood transfusions), and increased renal excretion (e.g., renal transplantation, cyclosporine therapy, cisplatin therapy, aminoglycoside therapy, amphotericin B therapy, capreomycin therapy, viomycin therapy, diuretic administration, pentamindine therapy, theophylline toxicity, and colony stimulation factor therapy).

In addition, manganese is an essential trace element for humans. Manganese acts as an activator of enzymes and as a components of oxidoreductase, hydrolase, and ligase metalloenzymes such as diamine oxidase and pyruvate carboxylase. Such enzymes are involved in oxidative phosphorylation, fatty acid, mucopolysaccharide, and cholesterol metabolism. Deficiencies of manganese causes skeletal, central nervous, and gonadal manifestations as well as bleeding disorders due to increased prothrombin times. Metal toxicity due to manganese excess results in encephalitis-like syndrome, Parkinson-like syndrome, psychosis, and pneumoconiosis.

The identification and isolation of receptors for magnesium and manganese would allow for the pharmacological, physiological and genetic modulation of magnesium levels, and magnesium-modulated pathways. For example, availability of the magnesium receptor would permit the screening for high affinity agonists, antagonists and inverse agonists that could be used as modulators of magnesium and manganese levels, and pathways.

### BRIEF SUMMARY OF THE INVENTION

We now report the identification of the mammalian magnesium sensor—a novel GPCR expressed in the principal centers of magnesium control (e.g. kidneys and thyroid). Such receptors are useful for assaying for compounds that modulate magnesium and manganese sensing and metabolism, for treatment of magnesium and manganese-related disease states and disorders of magnesium and manganese metabolism. The present invention also provides nucleic acid and amino acid sequences for the receptors of the invention.

In one aspect, the present invention provides a method of identifying a compound that modulates magnesium or manganese signal transduction in cells, the method comprising the steps of (i) contacting the compound with a receptor comprising a R5.24 polypeptide, wherein the R5.24 polypeptide is encoded by a nucleotide sequence that hybridizes under highly stringent hybridization conditions to a nucleotide sequence encoding an amino acid sequence of SEQ ID NO:2, 4, or 6; and (ii) determining the functional effect of the compound upon the receptor, thereby identifying a compound that modulates signal transduction by the receptor.

In one embodiment, the R5.24 polypeptide has an amino acid sequence of SEQ ID NO:2, 4, or 6. In another embodiment, wherein the receptor is recombinant. In another embodiment, the receptor has G protein coupled receptor activity.

In one embodiment, the functional effect is measured in vitro. In another embodiment, the functional effect is a physical effect. In another embodiment, the functional effect is determined by measuring ligand binding to the receptor. In another embodiment, the functional effect is a chemical or phenotypic effect. In another embodiment, the functional effect is determined by measuring changes in intracellular cAMP, IP3, or Ca2+.

In one embodiment, the receptor is linked to a solid phase. In another embodiment, the functional effect is determined by measuring binding of a compound to the receptor.

In one embodiment, the functional effect is determined by measuring binding of a compound to the extracellular domain of the receptor.

In one embodiment, the receptor is expressed in a cell or cell membrane.

In one embodiment, the cell is a mammalian cell. In another embodiment, the cell is a human cell.

### BRIEF DESCRIPTION OF THE DRAWINGS

FIG. 1. Activity assays for R5.24 receptor

Left panels: cells expressing R5.24 and stimulated with control solutions—>no responses. Middle panels: cells expressing R5.24 respond when stimulated with magnesium or manganese. Right panels: cells without the receptor do not respond to magnesium or manganese.

FIG. 2. In situ hybridization to thyroid tissue

Note strong expression in subsets of cells. EST analysis also reveals strong representation in kidney

FIGS. 3-5. Nucleotide and amino acid sequences for human, rat and mouse R5.24

### DETAILED DESCRIPTION OF THE INVENTION INTRODUCTION

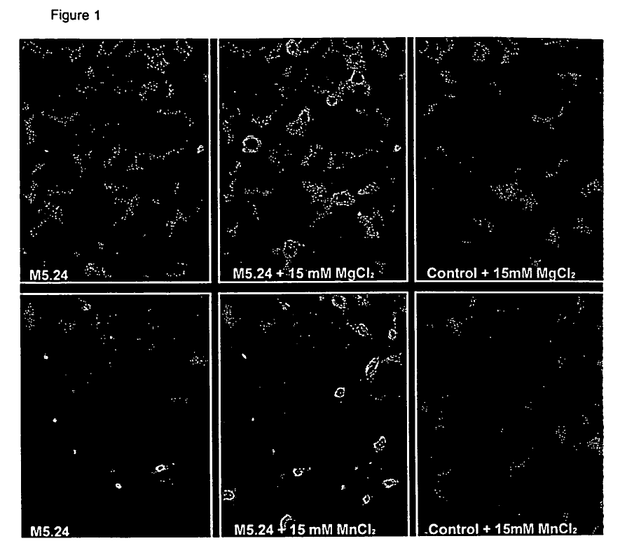

To date, there are many GPCRs for peptides, hormones, small metabolites, neurotransmitters, odorant and taste molecules, however there is only a single GPCR that is selective for an ion, namely the calcium-sensing receptor (CaSR). Although magnesium is also one of the four most abundant ions in animal physiology (together with sodium, potassium and calcium), and a component of over 300 biochemical reactions, the cellular system that senses and modulates magnesium levels is not known (see, e.g., Hofer and Brown (2003) Nature reviews 4, 530-538; Nelson et al (2001) Cell 106, 381-390; Nelson et al (2002) Nature 416, 199-202; Zhao et al (2003) Cell 115, 255-266). We reasoned that given the importance of magnesium, there should be a receptor responsible for its homeostasis. The CaSR is a member of the family C of GPCRs characterized by the presence of a large extracellular ligand binding domain. Therefore, we searched and tested candidate (orphan) GPCRs for activation by magnesium and/or manganese. Using cells lines expressing modified G proteins optimized to couple a wide range of GPCRs to calcium signals, we assayed several family C GPCRs, including the orphan GPCR R5.24, for responses to magnesium/manganese. FIG. 1 demonstrates that R5.24 functions as a magnesium/manganese receptor.

Candidate receptors are expressed in human embryonic kidney (HEK) cells containing, e.g., the Gα₁₆-Gα_{z} and/or Gα₁₅ promiscuous G proteins (Offermanns et al., J. Biol. Chem. 270:15175-15180 (1995); Mody et al., Mol. Pharmacol. 57:13-23 (2000)), and assayed for stimulus-evoked changes in intracellular calcium. In this system, receptor activation leads to activation of phospholipase Cβ (PLC-β and release of calcium from internal stores, which can be monitored at the single-cell level using calcium-indicator dyes (Chandrashekar et al., Cell 100:703-711 (2000); Nelson et al., Cell 106:381-390 (2001); Tsien et al., Cell Calcium 6:145-157 (1985)).

The present invention provides magnesium and manganese receptors comprising members of the C family of G-protein coupled receptors. When expressed in a cell, the polypeptide transduces signal in response to the magnesium or manganese ligands. These nucleic acids and proteins encoding the receptors provide for the pharmacological, physiological and genetic modulation of magnesium levels, and magnesium-modulated pathways. For example, availability of the magnesium receptor would permit the screening for high affinity agonists, antagonists and inverse agonists that could be used as modulators of magnesium levels, and pathways.

Such modulators would be useful for treating hypo- and hypermagnesium and manganese syndromes, e.g., cardiac arrhythmias, disturbances in conduction, ventricular fibrillation, cardiac arrest, anorexia, nausea, vomiting, lethargy, paresthesia, muscular cramps, irritability, and mental confusion; hypermagnesemia syndromes in infants born to pre-eclamptic and eclamptic mothers and subjects with adrenal insufficiency, renal disease, and rhabdomyolysis; hypomagnesemia syndromes in subjects with familial malabsorption, malnutrition, total parenteral nutrition, malabsorption syndromes (e.g., enteric fistulas, Whipple's disease, intestinal lymphoma, biliary diversion, giardiasis, and short bowel syndrome), specific absorptive defects, prolonged diarrhea, pancreatitis, endocrine disorders (e.g., hyperparathyroidism, hypoparathyroidism, hyperthyroidism, primary hyperaldosteronism, Bartter's syndrome, Gitelman's syndrome, and diabetic and alcoholic ketoacidosis), cellular uptake or redistribution (e.g., administration of epinephrine, massive blood transfusions), and increased renal excretion (e.g., renal transplantation, cyclosporine therapy, cisplatin therapy, aminoglycoside therapy, amphotericin B therapy, capreomycin therapy, viomycin therapy, diuretic administration, pentamindine therapy, theophylline toxicity, and colony stimulation factor therapy); disease related to oxidoreductase, hydrolase, and ligase metalloenzymes such as diamine oxidase and pyruvate carboxylase; diseases related to oxidative phosphorylation, fatty acid, mucopolysaccharide, and cholesterol metabolism; bleeding disorders due to increased prothrombin times; metal toxicity; encephalitis-like syndrome; Parkinson-like syndrome, psychosis, and pneumoconiosis.

GPCRs can be used in assays, e.g., to measure changes in ligand binding, G-protein binding, regulatory molecule binding, ion concentration, membrane potential, current flow, ion flux, transcription, signal transduction, receptor-ligand interactions, neurotransmitter and hormone release; and second messenger concentrations, in vitro, in vivo, and ex vivo. In one embodiment, a Mg receptor can be used as an indirect reporter via attachment to a second reporter molecule such as green fluorescent protein (see, e.g., Mistili & Spector, Nature Biotechnology 15:961-964 (1997)). In another embodiment, a Mg receptor is recombinantly expressed in cells, and modulation of signal transduction via GPCR activity is assayed by measuring changes in Ca2+ levels.

Methods of assaying for modulators include in vitro ligand binding assays using Mg receptors, portions thereof such as the extracellular domain, or chimeric proteins comprising one or more domains of the receptor, and in in vivo (cell-based and animal) assays such as oocyte Mg receptor expression; tissue culture cell Mg receptor expression; transcriptional activation; phosphorylation and dephosphorylation of GPCRs; G-protein binding to GPCRs; ligand binding assays; voltage, membrane potential and conductance changes; ion flux assays; changes in intracellular second messengers such as cAMP and inositol triphosphate; changes in intracellular calcium levels; and neurotransmitter release.

Definitions

The terms “GPCR R5.24,” “Mg receptor” and “Mn receptor” or a nucleic acid encoding “GPCR R5.24,” “Mg receptor” and “Mn receptor” refer to nucleic acid and polypeptide polymorphic variants, alleles, mutants, and interspecies homologs that are members of the C family of G protein coupled receptors (including the calcium sensing receptor CaSR) and: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an amino acid sequence encoded by SEQ ID NO:1, 2, 3, 7, 8, 9, 15, 18, 20, 23, 25, 27, or 30; (2) bind to antibodies, e.g., polyclonal antibodies, raised against an immunogen comprising an amino acid sequence encoded by SEQ ID NO:1, 2, 3, 7, 8, 9, 15, 18, 20, 23, 25, 27, or 30, and conservatively modified variants thereof; (3) specifically hybridize under stringent hybridization conditions to an anti-sense strand corresponding to a nucleic acid sequence encoding a R5.24 protein, e.g., SEQ ID NO:4, 5, 6, 10, 11, 12, 13, 14, 16, 17, 19, 21, 22, 24, 26, 28, or 29, and conservatively modified variants thereof; (4) have a nucleic acid sequence that has greater than about 60% sequence identity, 65%, 70%, 75%, 80%, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to SEQ ID NO:4, 5, 6, 10, 11, 12, 13, 14, 16, 17, 19, 21, 22, 24, 26, or 28, or 29. The R5.24 receptor has the ability to recognize magnesium and manganese ligands, as described herein. These molecules are examples of compounds that “modulate signal transduction” by acting as ligands for the R5.24G protein coupled receptor.

R5.24 proteins have “G-protein coupled receptor activity,” e.g., they bind to G-proteins in response to extracellular stimuli, such as ligand binding (e.g., Mg or Mn molecules), and promote production of second messengers such as IP3, cAMP, and Ca2+ via stimulation of enzymes such as phospholipase C and adenylate cyclase. Such activity can be measured in a heterologous cell, by coupling a GPCR (or a chimeric GPCR) to either a G-protein or promiscuous G-protein such as Gα₁₅, and an enzyme such as PLC, and measuring increases in intracellular calcium using (Offermans & Simon, J. Biol. Chem. 270:15175-15180 (1995)). Receptor activity can be effectively measured, e.g., by recording ligand-induced changes in [Ca²⁺]ᵢ using fluorescent Ca²⁺-indicator dyes and fluorometric imaging.

Such GPCRs have transmembrane, extracellular and cytoplasmic domains that can be structurally identified using methods known to those of skill in the art, such as sequence analysis programs that identify hydrophobic and hydrophilic domains (see, e.g., Kyte & Doolittle, J. Mol. Biol. 157:105-132 (1982)). Such domains are useful for making chimeric proteins and for in vitro assays of the invention (see, e.g., WO 94/05695 and U.S. Pat. No. 5,508,384). In particular, the R5.24 receptor of the invention is a member of the C family of GPCRs, characterized by the presence of a large extracellular binding domain. This domain can be used to make chimeric proteins for in vivo and in vitro assays. The extracellular domain alone is also useful for in vitro binding assays.

The phrase “functional effects” in the context of assays for testing compounds that modulate activity (e.g., signal transduction) of a Mg receptor or protein of the invention includes the determination of a parameter that is indirectly or directly under the influence of a GPCR or Mg/Mn receptor, e.g., a physical, phenotypic, or chemical effect, such as the ability to transduce a cellular signal in response to external stimuli such as ligand binding, or the ability to bind a ligand. It includes binding activity and signal transduction. “Functional effects” include in vitro, in vivo, and ex vivo activities.

By “determining the functional effect” is meant assaying for a compound that increases or decreases a parameter that is indirectly or directly under the influence of aR5.24 GPCR protein or a Mg receptor comprising one or more R5.24 proteins, e.g., physical and chemical or phenotypic effect. Such functional effects can be measured by any means known to those skilled in the art, e.g., changes in spectroscopic characteristics (e.g., fluorescence, absorbance, refractive index); hydrodynamic (e.g., shape); chromatographic; or solubility properties for the protein; measuring inducible markers or transcriptional activation of the protein; measuring binding activity or binding assays, e.g., binding to antibodies; measuring changes in ligand binding activity or analogs thereof, either naturally occurring or synthetic; measuring cellular proliferation; measuring cell surface marker expression, measurement of changes in protein levels for R5.24-associated sequences; measurement of RNA stability; G-protein binding; GPCR phosphorylation or dephosphorylation; signal transduction, e.g., receptor-ligand interactions, second messenger concentrations (e.g., cAMP, cGMP, IP3, PI, or intracellular Ca²⁺); neurotransmitter release; hormone release; voltage, membrane potential and conductance changes; ion flux; regulatory molecule binding; identification of downstream or reporter gene expression (CAT, luciferase, β-gal, GFP and the like), e.g., via chemiluminescence, fluorescence, colorimetric reactions, antibody binding, and inducible markers.

“Inhibitors,” “activators,” and “modulators” of R5.24 polynucleotide and polypeptide sequences and R5.24 receptors are used to refer to activating, inhibitory, or modulating molecules identified using in vitro and in vivo assays of R5.24 polynucleotide and polypeptide sequences and R5.24 family receptors, including monomeric, homodimeric, and heterodimeric receptors. Inhibitors are compounds that, e.g., bind to, partially or totally block activity, decrease, prevent, delay activation, inactivate, desensitize, or down regulate the activity or expression of the R5.24 receptor, e.g., antagonists. “Activators” are compounds that increase, open, activate, facilitate, enhance activation, sensitize, agonize, or up regulate a R5.24 receptor, e.g., agonists. Inhibitors, activators, or modulators also include genetically modified versions of R5.24 receptors, e.g., versions with altered activity, as well as naturally occurring and synthetic ligands, antagonists, agonists, antibodies, antisense molecules, ribozymes, small chemical molecules and the like. Such assays for inhibitors and activators include, e.g., expressing R5.24 receptors in vitro, in cells, or cell membranes, applying putative modulator compounds, and then determining the functional effects on activity, as described above. In one embodiment, a R5.24 receptor has the ability to recognize a Mg or Mn molecule, as described herein. These molecules are examples of compounds that modulate signal transduction by acting as extracellular ligands for the G protein coupled receptor and activating the receptor. In other embodiments, compounds that modulate signal transduction are molecules that act as intracellular ligands of the receptor, or inhibit or activate binding of an extracellular ligand, or inhibit or activate binding of intracellular ligands of the receptor.

Samples or assays comprising R5.24 receptors are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a R5.24 receptor is achieved when the activity value relative to the control is about 80%, preferably 50%, more preferably 25-0%. Activation of a R5.24 receptor is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, RNAi or snRNA molecule etc., to be tested for the capacity to directly or indirectly modulation magnesium and manganese sensing and metabolism. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

A “heterodimer” is a dimer receptor comprising two different molecules, e.g., two different polypeptides, where the molecules are associated via either covalent, e.g., through a linker or a chemical bond, or non-covalent, e.g., ionic, van der Waals, electrostatic, or hydrogen bonds linkages. A “homodimer” is a dimer receptor comprising two of the same molecules linked either covalently or noncovalently as described above. A monomer is a single molecule receptor.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequences SEQ ID NO:1-6), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, enantiomers (D- and L-forms), and achiral amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., extracellular domains, transmembrane domains, and cytoplasmic domains. Domains are portions of a polypeptide that form a compact unit of the polypeptide and are typically 15 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

“Nucleic acid” refers to deoxyribonucleotides or ribonucleotides and polymers thereof in either single- or double-stranded form, as well as the complements of any such sequence. Also included are DNA, cDNA, RNA, polynucleotides, nucleotides, and the like. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al., PCR Protocols, A Guide to Methods and Applications (1990).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to a R5.24 protein comprising a sequence of or encoded by SEQ ID NO:1-6, polymorphic variants, alleles, orthologs, and conservatively modified variants, or splice variants, or portions thereof, can be selected to obtain only those polyclonal antibodies that are specifically immunoreactive with R5.24 proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

Isolation of Nucleic Acids Encoding R5.24 Proteins

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

R5.24 nucleic acids, polymorphic variants, orthologs, and alleles that are substantially identical to an amino acid sequences disclosed herein can be isolated using R5.24 nucleic acid probes and oligonucleotides under stringent hybridization conditions, by screening libraries. Alternatively, expression libraries can be used to clone R5.24 protein, polymorphic variants, orthologs, and alleles by detecting expressed homologs immunologically with antisera or purified antibodies made against human R5.24 or portions thereof.

To make a cDNA library, one should choose a source that is rich in R5.24 RNA, e.g., kidney or thyroid. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating R5.24 nucleic acid and its orthologs, alleles, mutants, polymorphic variants, and conservatively modified variants combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of human R5.24 directly from mRNA, from cDNA, from genomic libraries or cDNA libraries. Degenerate oligonucleotides can be designed to amplify R5.24 homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of R5.24 encoding mRNA in physiological samples, for nucleic acid sequencing, or for other purposes. Genes amplified by the PCR reaction can be purified from agarose gels and cloned into an appropriate vector.

Gene expression of R5.24 can also be analyzed by techniques known in the art, e.g., reverse transcription and amplification of mRNA, isolation of total RNA or poly A+ RNA, northern blotting, dot blotting, in situ hybridization, RNase protection, high density polynucleotide array technology, e.g., and the like.

Nucleic acids encoding R5.24 protein can be used with high density oligonucleotide array technology (e.g., GeneChip™) to identify R5.24 protein, orthologs, alleles, conservatively modified variants, and polymorphic variants in this invention (see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998)).

The gene for R5.24 is typically cloned into intermediate vectors before transformation into prokaryotic or eukaryotic cells for replication and/or expression. These intermediate vectors are typically prokaryote vectors, e.g., plasmids, or shuttle vectors.

Expression in Prokaryotes and Eukaryotes

To obtain high level expression of a cloned gene, such as those cDNAs encoding a R5.24 protein, one typically subclones R5.24 into an expression vector that contains a strong promoter to direct transcription, a transcription/translation terminator, and if for a nucleic acid encoding a protein, a ribosome binding site for translational initiation. The R5.24 nucleic acids can be co-expressed or separately expressed, preferably co-expressed on the same or a different vector. Suitable bacterial promoters are well known in the art and described, e.g., in Sambrook et al., and Ausubel et al, supra. Bacterial expression systems for expressing the R5.24 protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In one preferred embodiment, retroviral expression systems are used in the present invention.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the R5.24 encoding nucleic acid in host cells. A typical expression cassette thus contains a promoter operably linked to the nucleic acid sequence encoding R5.24 and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, β-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

In one embodiment, the vectors of the invention have a regulatable promoter, e.g., tet-regulated systems and the RU-486 system (see, e.g., Gossen & Bujard, Proc. Nat'l Acad. Sci. USA 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a R5.24 encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of R5.24 protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing R5.24.

After the expression vector is introduced into the cells, the transfected cells are cultured under conditions favoring expression of R5.24, which is recovered from the culture using standard techniques identified below.

Purification of R5.24 Polypeptides

Either naturally occurring or recombinant R5.24 polypeptides or R5.243-comprising receptors can be purified for use in functional assays. Naturally occurring R5.24 proteins or R5.24-comprising receptors can be purified, e.g., from human tissue. Recombinant R5.24 proteins or R5.24-comprising receptors can be purified from any suitable expression system.

The R5.24 protein may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant R5.24 protein is being purified. For example, proteins having established molecular adhesion properties can be reversible fused to the R5.24 protein. With the appropriate ligand, R5.24 protein can be selectively adsorbed to a purification column and then freed from the column in a relatively pure form. The fused protein is then removed by enzymatic activity. Finally, R5.24 protein could be purified using immunoaffinity columns.

A. Purification of R5.24 from Recombinant Bacteria

Recombinant proteins are expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of R5.24 protein inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. Human R5.24 proteins are separated from other bacterial proteins by standard separation techniques, e.g., with Ni-NTA agarose resin.

Alternatively, it is possible to purify R5.24 protein from bacteria periplasm. After lysis of the bacteria, when the R5.24 protein is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying R5.24 Proteins

Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of the R5.24 proteins can be used to isolate it from proteins of greater and lesser size using ultrafiltration through membranes of different pore size (For example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

The R5.24 proteins can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Assays for Modulators of R5.24 Protein

A. Assays

Modulation of a R5.24-comprising receptor, and corresponding modulation of taste, can be assessed using a variety of in vitro and in vivo assays. Such assays can be used to test for inhibitors and activators of R5.24-comprising receptors, and, consequently, inhibitors and activators of magnesium and manganese sensing and metabolism. Modulators of R5.24-comprising receptors are tested using either recombinant or naturally occurring R5.24-comprising receptors, preferably human receptors.

Preferably, the R5.24-comprising receptor will have a sequence as encoded by a sequence provided herein or a conservatively modified variant thereof. Alternatively, the R5.24-comprising receptor of the assay will be derived from a eukaryote and include an amino acid subsequence having substantial amino acid sequence identity to the sequences provided herein or is encoded by a nucleotide sequence that hybridizes under stringent conditions (moderate or high) to a nucleotide sequence as described herein. Generally, the amino acid sequence identity will be at least 60%, preferably at least 65%, 70%, 75%, 80%, 85%, or 90%, most preferably at least 95%.

Measurement of Mg and Mn signal transduction or loss-of-Mg and Mn signal transduction phenotype on R5.24-comprising receptor or cell expressing the R5.24-comprising receptor, either recombinant or naturally occurring, can be performed using a variety of assays, in vitro, in vivo, and ex vivo, as described herein. A suitable physical, chemical or phenotypic change that affects activity or binding can be used to assess the influence of a test compound on the polypeptide of this invention. When the functional effects are determined using intact cells or animals, one can also measure a variety of effects such as, in the case of signal transduction, e.g., ligand binding, hormone release, transcriptional changes to both known and uncharacterized genetic markers (e.g., northern blots), changes in cell metabolism such as pH changes, and changes in intracellular second 2+messengers such as Ca²⁺, IP3, cGMP, or cAMP.

In Vitro Assays

Assays to identify compounds with R5.24-comprising receptor modulating activity can be performed in vitro. Such assays can use a full length R5.24-comprising receptor or a variant thereof, or a fragment of a R5.24-comprising receptor, such as an extracellular domain, fused to a heterologous protein to form a chimera. Purified recombinant or naturally occurring R5.24-comprising receptor can be used in the in vitro methods of the invention. In addition to purified R5.24-comprising receptor, the recombinant or naturally occurring R5.24-comprising receptor can be part of a cellular lysate or a cell membrane. As described below, the binding assay can be either solid state or soluble. Preferably, the protein or membrane is bound to a solid support, either covalently or non-covalently. Often, the in vitro assays of the invention are ligand binding or ligand affinity assays, either non-competitive or competitive (with known extracellular ligands as described herein, or with a known intracellular ligand GTP). Other in vitro assays include measuring changes in spectroscopic (e.g., fluorescence, absorbance, refractive index), hydrodynamic (e.g., shape), chromatographic, or solubility properties for the protein.

In one embodiment, a high throughput binding assay is performed in which the R5.24-comprising receptor or chimera comprising a fragment thereof is contacted with a potential modulator and incubated for a suitable amount of time. In one embodiment, the potential modulator is bound to a solid support, and the R5.24-comprising receptor is added. In another embodiment, the R5.24-comprising receptor is bound to a solid support. A wide variety of modulators can be used, as described below, including small organic molecules, peptides, antibodies, and R5.24-comprising receptor ligand analogs. A wide variety of assays can be used to identify R5.24-comprising receptor-modulator binding, including labeled protein-protein binding assays, electrophoretic mobility shifts, immunoassays, enzymatic assays such as phosphorylation assays, and the like. In some cases, the binding of the candidate modulator is determined through the use of competitive binding assays, where interference with binding of a known ligand is measured in the presence of a potential modulator. Ligands for R5.24-comprising receptors are provided herein. Either the modulator or the known ligand is bound first, and then the competitor is added. After the R5.24-comprising receptor is washed, interference with binding, either of the potential modulator or of the known ligand, is determined. Often, either the potential modulator or the known ligand is labeled.

Cell-based In Vivo Assays

In another embodiment, a R5.24-comprising receptor is expressed in a cell, and functional, e.g., physical and chemical or phenotypic, changes are assayed to identify R5.24-comprising receptor modulators. Cells expressing R5.24-comprising receptor can also be used in binding assays. Any suitable functional effect can be measured, as described herein. For example, ligand binding, G-protein binding, and GPCR signal transduction, e.g., changes in intracellular Ca²⁺ levels, are all suitable assays to identify potential modulators using a cell based system. Suitable cells for such cell based assays include both primary cells and cell lines, as described herein. The R5.24-comprising receptor can be naturally occurring or recombinant. Also, as described above, chimeric R5.24-comprising receptors with GPCR activity can be used in cell based assays. For example, the extracellular domain of an R5.24 protein can be fused to the transmembrane and/or cytoplasmic domain of a heterologous protein, preferably a heterologous GPCR. Such a chimeric GPCR would have GPCR activity and could be used in cell based assays of the invention.

In another embodiment, cellular R5.24 polypeptide levels are determined by measuring the level of protein or mRNA. The level of R5.24 protein or proteins related to R5.24 signal transduction are measured using immunoassays such as western blotting, ELISA and the like with an antibody that selectively binds to the R5.24-comprising receptor or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, R5.24-comprising receptor expression can be measured using a reporter gene system. Such a system can be devised using an R5.24 protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

In another embodiment, a functional effect related to GPCR signal transduction can be measured. An activated or inhibited R5.24-comprising G-coupled protein receptor will alter the properties of target enzymes, second messengers, channels, and other effector proteins. The examples include the activation of cGMP phosphodiesterase, adenylate cyclase, phospholipase C, IP3, and modulation of diverse channels by G proteins. Downstream consequences can also be examined such as generation of diacyl glycerol and IP3 by phospholipase C, and in turn, for calcium mobilization by IP3. Activated GPCR receptors become substrates for kinases that phosphorylate the C-terminal tail of the receptor (and possibly other sites as well). Thus, activators will promote the transfer of ³²P from gamma-labeled GTP to the receptor, which can be assayed with a scintillation counter. The phosphorylation of the C-terminal tail will promote the binding of arrestin-like proteins and will interfere with the binding of G-proteins. For a general review of GPCR signal transduction and methods of assaying signal transduction, see, e.g., Methods in Enzymology, vols. 237 and 238 (1994) and volume 96 (1983); Bourne et al., Nature 10:349:117-27 (1991); Bourne et al., Nature 348:125-32 (1990); Pitcher et al., Annu. Rev. Biochem. 67:653-92 (1998).

As described above, activation of some G-protein coupled receptors stimulates the formation of inositol triphosphate (IP3) through phospholipase C-mediated hydrolysis of phosphatidylinositol (Berridge & Irvine, Nature 312:315-21 (1984)). IP3 in turn stimulates the release of intracellular calcium ion stores. Thus, a change in cytoplasmic calcium ion levels, or a change in second messenger levels such as IP3 can be used to assess G-protein coupled receptor function. Cells expressing such G-protein coupled receptors may exhibit increased cytoplasmic calcium levels as a result of contribution from both intracellular stores and via activation of ion channels, in which case it may be desirable although not necessary to conduct such assays in calcium-free buffer, optionally supplemented with a chelating agent such as EGTA, to distinguish fluorescence response resulting from calcium release from internal stores.

In one example, R5.24-comprising receptor GPCR activity is measured by expressing a R5.24-comprising receptor in a heterologous cell with a promiscuous G-protein that links the receptor to a phospholipase C signal transduction pathway (see Offermanns & Simon, J. Biol. Chem. 270:15175-15180 (1995)). Modulation of signal transduction is assayed by measuring changes in intracellular Ca²⁺ levels, which change in response to modulation of the GPCR signal transduction pathway via administration of a molecule that associates with an R5.24-comprising receptor. Changes in Ca²⁺ levels are optionally measured using fluorescent Ca²⁺ indicator dyes and fluorometric imaging.

In another example, phosphatidyl inositol (PI) hydrolysis can be analyzed according to U.S. Pat. No. 5,436,128, herein incorporated by reference. Briefly, the assay involves labeling of cells with ³H-myoinositol for 48 or more hrs. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist to cpm in the presence of buffer control. Likewise, fold inhibition is determined by calculating the ratio of cpm in the presence of antagonist to cpm in the presence of buffer control (which may or may not contain an agonist).

Other assays can involve determining the activity of receptors which, when activated, result in a change in the level of intracellular cyclic nucleotides, e.g., cAMP or cGMP, by activating or inhibiting enzymes such as adenylate cyclase. In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay.

In one example, the changes in intracellular cAMP or cGMP can be measured using immunoassays. The method described in Offermanns & Simon, J. Biol. Chem. 270:15175-15180 (1995) may be used to determine the level of cAMP. Also, the method described in Felley-Bosco et al., Am. J. Resp. Cell and Mol. Biol. 11:159-164 (1994) may be used to determine the level of cGMP. Further, an assay kit for measuring cAMP and/or cGMP is described in U.S. Pat. No. 4,115,538, herein incorporated by reference.

In one example, assays for G-protein coupled receptor activity include cells that are loaded with ion or voltage sensitive dyes to report receptor activity. Assays for determining activity of such receptors can also use known agonists and antagonists for other G-protein coupled receptors as negative or positive controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. For G-protein coupled receptors, promiscuous G-proteins such as Gα₁₅ and Gα₁₆ can be used in the assay of choice (Wilkie et al., Proc. Nat'l Acad. Sci. USA 88:10049-10053 (1991)). Such promiscuous G-proteins allow coupling of a wide range of receptors.

Animal Models

Animal models also find use in screening for modulators of Mg and Mn sensing and metabolism. Transgenic animal technology including gene knockout technology, for example as a result of homologous recombination with an appropriate gene targeting vector, or gene overexpression, will result in the absence or increased expression of the R5.24-comprising receptor or components thereof. When desired, tissue-specific expression or knockout of the R5.24-comprising receptors or components thereof may be necessary. Transgenic animals generated by such methods find use as animal models of Mg and Mn modulation and are additionally useful in screening for modulators of Mg and Mn sensing and metabolism to identify therapeutic compounds for treatment of disease.

B. Modulators

The compounds tested as modulators of R5.24-comprising receptors can be any small organic molecule, or a biological entity, such as a protein, e.g., an antibody or peptide, a sugar, a nucleic acid, e.g., an antisense oligonucleotide, RNAi, snRNA, or a ribozyme, or a lipid. Alternatively, modulators can be genetically altered versions of a R5.24-comprising taste receptor. Typically, test compounds will be small organic molecules, peptides, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

C. Solid State and Soluble High Throughput Assays

In one embodiment the invention provides soluble assays using a R5.24-comprising receptor, or a cell or tissue expressing a R5.24-comprising receptor, either naturally occurring or recombinant. In another embodiment, the invention provides solid phase based in vitro assays in a high throughput format, where the R5.24-comprising receptor is attached to a solid phase substrate. Any one of the assays described herein can be adapted for high throughput screening, e.g., ligand binding, cellular proliferation, cell surface marker flux, e.g., screening, radiolabeled GTP binding, second messenger flux, e.g., Ca²⁺, IP3, cGMP, or cAMP, cytokine production, etc.

In the high throughput assays of the invention, either soluble or solid state, it is possible to screen up to several thousand different modulators or ligands in a single day. This methodology can be used for R5.24-comprising receptors in vitro, or for cell-based or membrane-based assays comprising R5.24-comprising receptors. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

Immunological Detection of R5.24-Comprising Receptors

In addition to the detection of R5.24 genes and gene expression using nucleic acid hybridization technology, one can also use immunoassays to detect R5.24-comprising taste receptors of the invention. Such assays are useful for screening for modulators of R5.24-comprising receptors, as well as for therapeutic and diagnostic applications. Immunoassays can be used to qualitatively or quantitatively analyze R5.24-comprising receptors. A general overview of the applicable technology can be found in Harlow & Lane, Antibodies: A Laboratory Manual (1988).

A. Production of Antibodies

Methods of producing polyclonal and monoclonal antibodies that react specifically with the R5.24 proteins and R5.24-comprising receptors are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A number of immunogens comprising portions of R5.24-comprising receptor may be used to produce antibodies specifically reactive with R5.24 protein. For example, recombinant R5.24 protein or an antigenic fragment thereof, can be isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-R5.24 or R5.24-comprising receptor proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular R5.24-comprising receptor ortholog, such as human R5.24-comprising receptor, can also be made, by subtracting out other cross-reacting orthologs from a species such as a non-human mammal.

Once the specific antibodies against R5.24-comprising receptors are available, the protein can be detected by a variety of immunoassay methods. In addition, the antibody can be used therapeutically as a R5.24-comprising receptor modulators. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra.

B. Immunological Binding Assays

R5.24-comprising receptors can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case the R5.24-comprising receptor or antigenic subsequence thereof). The antibody (e.g., anti-R5.24-comprising receptor) may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled R5.24-comprising receptor or a labeled anti-R5.24-comprising receptor antibody. Alternatively, the labeling agent may be a third moiety, such a secondary antibody, that specifically binds to the antibody/R5.24-comprising receptor complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111: 1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Non-Competitive Assay Formats

Immunoassays for detecting R5.24-comprising receptors in samples may be either competitive or noncompetitive. Noncompetitive immunoassays are assays in which the amount of antigen is directly measured. In one preferred “sandwich” assay, for example, the anti-R5.24-comprising receptor antibodies can be bound directly to a solid substrate on which they are immobilized. These immobilized antibodies then capture R5.24-comprising receptors present in the test sample. R5.24-comprising receptors thus immobilized are then bound by a labeling agent, such as a second R5.24-comprising receptor antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Competitive Assay Formats

In competitive assays, the amount of R5.24-comprising receptor present in the sample is measured indirectly by measuring the amount of a known, added (exogenous) R5.24-comprising receptor displaced (competed away) from an anti-R5.24-comprising receptor antibody by the unknown R5.24-comprising receptor present in a sample. In one competitive assay, a known amount of R5.24-comprising receptor is added to a sample and the sample is then contacted with an antibody that specifically binds to a R5.24-comprising receptor. The amount of exogenous R5.24-comprising receptor bound to the antibody is inversely proportional to the concentration of R5.24-comprising receptor present in the sample. In a particularly preferred embodiment, the antibody is immobilized on a solid substrate. The amount of R5.24-comprising receptor bound to the antibody may be determined either by measuring the amount of R5.24-comprising receptor present in a R5.24-comprising receptor/antibody complex, or alternatively by measuring the amount of remaining uncomplexed protein. The amount of R5.24-comprising receptor may be detected by providing a labeled R5.24-comprising receptor molecule.

A hapten inhibition assay is another preferred competitive assay. In this assay the known R5.24-comprising receptor is immobilized on a solid substrate. A known amount of anti-R5.24-comprising receptor antibody is added to the sample, and the sample is then contacted with the immobilized R5.24-comprising receptor. The amount of anti-R5.24-comprising receptor antibody bound to the known immobilized R5.24-comprising receptor is inversely proportional to the amount of R5.24-comprising receptor present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Cross-Reactivity Determinations

Immunoassays in the competitive binding format can also be used for crossreactivity determinations. For example, a R5.24-comprising receptor can be immobilized to a solid support. Proteins (e.g., R5.24-comprising receptors and homologs) are added to the assay that compete for binding of the antisera to the immobilized antigen. The ability of the added proteins to compete for binding of the antisera to the immobilized protein is compared to the ability of the R5.24-comprising receptor to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of a R5.24-comprising receptor, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the R5.24-comprising receptor that is required to inhibit 50% of binding, then the second protein is said to specifically bind to the polyclonal antibodies generated to a R5.24-comprising receptor immunogen.

Other Assay Formats

Western blot (immunoblot) analysis is used to detect and quantify the presence of R5.24-comprising receptors in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind R5.24-comprising receptors. The anti-R5.24-comprising receptor antibodies specifically bind to the R5.24-comprising receptor on the solid support. These antibodies may be directly labeled or alternatively may be subsequently detected using labeled antibodies (e.g., labeled sheep anti-mouse antibodies) that specifically bind to the anti-R5.24-comprising receptor antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

Reduction of Non-Specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize R5.24-comprising receptors, or secondary antibodies that recognize anti-R5.24-comprising receptor.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Pharmaceutical Compositions and Administration

The compounds identified by the assays of the invention can be used to treat disease related to magnesium and manganese excess and deficiency. Furthermore, the R5.24 polypeptide and nucleic acids of the invention can also be used to treat such diseases. Pharmaceutically acceptable carriers are determined in part by the particular composition being administered (e.g., nucleic acid, oligonucleotide, protein, peptide, small organic molecule, lipid, carbohydrate, particle, or transduced cell), as well as by the particular method used to administer the composition. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, transdermal application, or rectal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions owing to diminished or aberrant expression of a R5.24-comprising receptor, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

Cellular Transfection and Gene Therapy

The present invention provides the nucleic acids of R5.24-comprising receptors for the transfection of cells in vitro and in vivo. These nucleic acids can be inserted into any of a number of well-known vectors for the transfection of target cells and organisms as described below, for the treatment of disease related to magnesium and manganese excess and deficiency. The nucleic acids are transfected into cells, ex vivo or in vivo, through the interaction of the vector and the target cell. The nucleic acid, under the control of a promoter, then expresses a R5.24-comprising receptor of the present invention, by co-expressing two members of the R5.24 family, thereby mitigating the effects of absent, partial inactivation, or abnormal expression of a R5.24-comprising receptor. The compositions are administered to a patient in an amount sufficient to elicit a therapeutic response in the patient. An amount adequate to accomplish this is defined as “therapeutically effective dose or amount.”

Such gene therapy procedures have been used to correct acquired and inherited genetic defects and other diseases in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11:167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology (Doerfler & Bohm eds., 1995); and Yu et al., Gene Therapy 1: 13-26 (1994)).

Kits

Human R5.24 and its homologs are useful tools for diagnosis of disease related to manganese and magnesium excess and deficiency. Human R5.24-specific reagents that specifically hybridize to R5.24 nucleic acid, such as R5.24 probes and primers, and R5.24-specific reagents that specifically bind to the R5.24 protein, e.g., R5.24 antibodies, are used to examine expression and identify mutations, under expression, and overexpression.

Nucleic acid assays for the presence of R5.24 DNA and RNA in a sample include numerous techniques are known to those skilled in the art, such as Southern analysis, northern analysis, dot blots, RNase protection, S1 analysis, amplification techniques such as PCR and LCR, and in situ hybridization. In in situ hybridization, for example, the target nucleic acid is liberated from its cellular surroundings in such as to be available for hybridization within the cell while preserving the cellular morphology for subsequent interpretation and analysis. The following articles provide an overview of the art of in situ hybridization: Singer et al., Biotechniques 4:230-250 (1986); Haase et al., Methods in Virology, vol. VII, pp. 189-226 (1984); and Nucleic Acid Hybridization: A Practical Approach (Hames et al., eds. 1987). In addition, CNG2B protein can be detected with the various immunoassay techniques described above. The test sample is typically compared to both a positive control (e.g., a sample expressing recombinant CNG2B monomers) and a negative control.

The present invention also provides for kits for screening modulators of the R5.24 polypeptides of the invention. Such kits can be prepared from readily available materials and reagents. For example, such kits can comprise any one or more of the following materials: R5.24 polypeptides or extracellular domains or cells expressing R5.24 polypeptides, reaction tubes, and instructions for testing the activities of R5.24. A wide variety of kits and components can be prepared according to the present invention, depending upon the intended user of the kit and the particular needs of the user. For example, the kit can be tailored for in vitro or in vivo assays for measuring the activity of a R5.24 receptor.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are offered to illustrate, but not to limit the claimed invention.

### Example 1

### R5.24 is a Magnesium Receptor

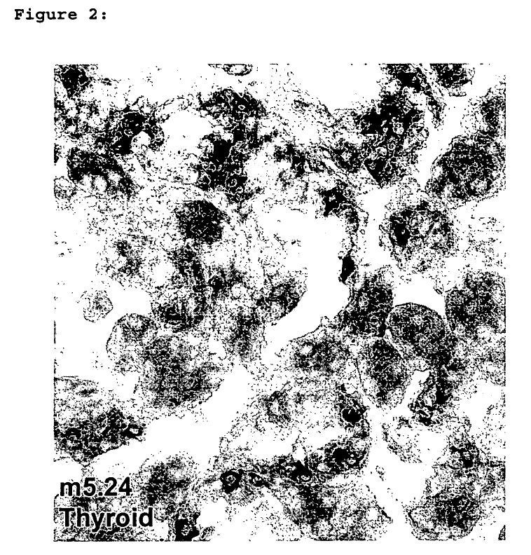

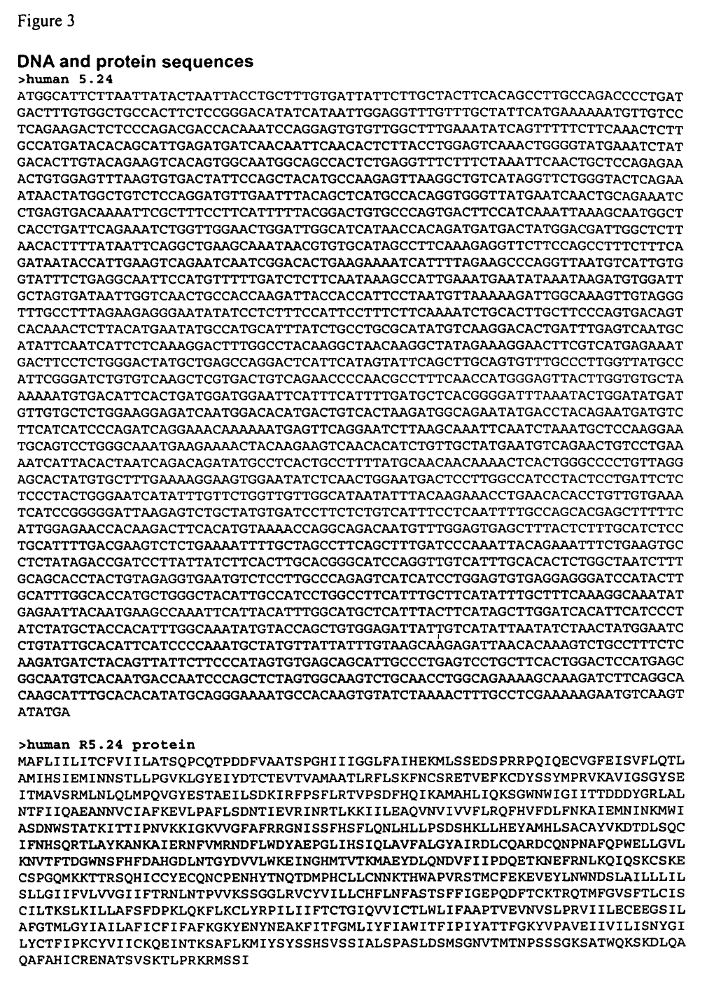

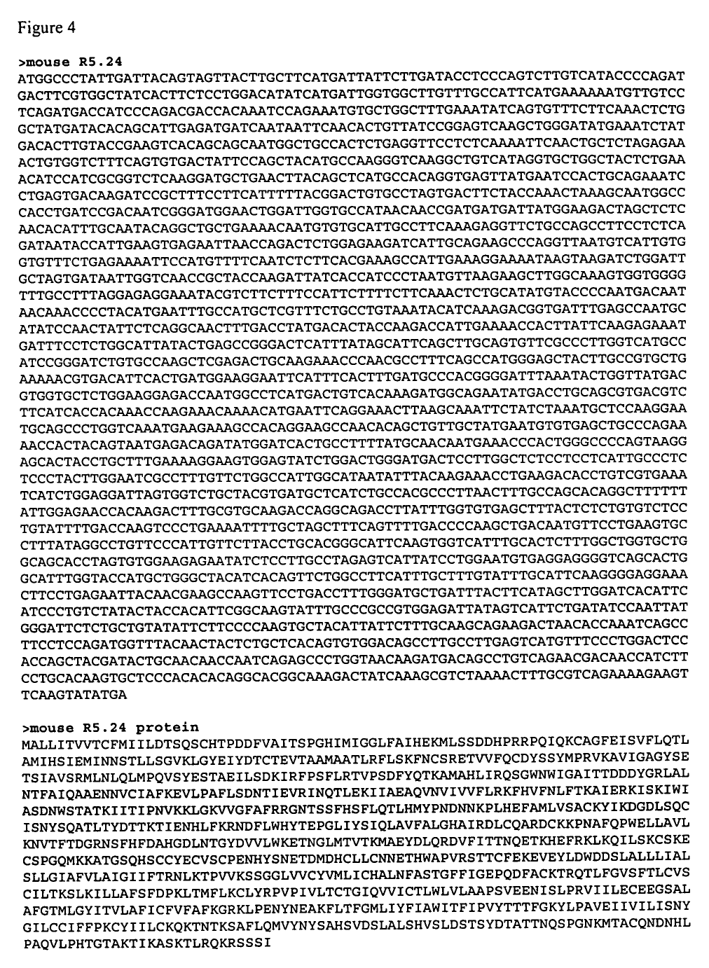

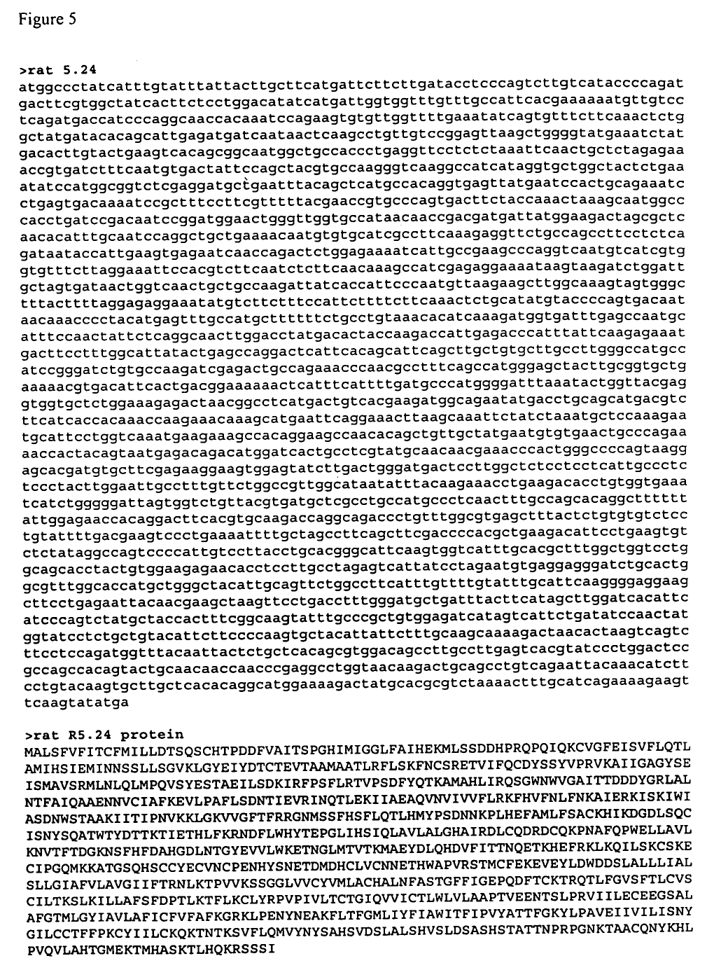

Using cells lines expressing modified G proteins optimized to couple a wide range of GPCRs to calcium signals, we assayed several family C GPCRs, including the orphan GPCR R5.24, for responses to magnesium/manganese. FIG. 1 demonstrates that R5.24 functions as a magnesium/manganese receptor. FIG. 2 shows in situ hybridization to thyroid tissues. EST analysis also shows strong expression in the kidney.

1. A method of identifying a compound that modulates magnesium or manganese signal transduction in cells, the method comprising the steps of
(i) contacting the compound with a receptor comprising a R5.24 polypeptide, wherein the R5.24 polypeptide is encoded by a nucleotide sequence that hybridizes under highly stringent hybridization conditions to a nucleotide sequence encoding an amino acid sequence of SEQ ID NO:2, 4, or 6; and
(ii) determining the functional effect of the compound upon the receptor, thereby identifying a compound that modulates signal transduction by the receptor.

2. The method of claim 1, wherein the R5.24 polypeptide has an amino acid sequence of SEQ ID NO:2, 4, or 6.

3. The method of claim 1, wherein the receptor is recombinant.

4. The method of claim 1, wherein the receptor has G protein coupled receptor activity.

5. The method of claim 1, wherein the functional effect is measured in vitro.

6. The method of claim 5, wherein the functional effect is a physical effect.

7. The method of claim 5, wherein the receptor is linked to a solid phase.

8. The method of claim 5, wherein the functional effect is determined by measuring binding of a compound to the receptor.

9. The method of claim 8, wherein the functional effect is determined by measuring binding of a compound to the extracellular domain of the receptor.

10. The method of claim 1, wherein the receptor is expressed in a cell or cell membrane.

11. The method of claim 10, wherein the functional effect is a physical effect.

12. The method of claim 11, wherein the functional effect is determined by measuring ligand binding to the receptor.

13. The method of claim 12, wherein the functional effect is determined by measuring binding of a compound to the extracellular domain of the receptor.

14. The method of claim 10, wherein the functional effect is a chemical or phenotypic effect.

15. The method of claim 14, wherein the functional effect is determined by measuring changes in intracellular cAMP, IP3, or Ca²⁺.

16. The method of claim 10, wherein the cell is a mammalian cell.

17. The method of claim 16, wherein the cell is a human cell.

18. A method of identifying a compound that modulates magnesium or manganese signal transduction in cells, the method comprising the steps of
(i) contacting the compound with cell expressing a receptor comprising a R5.24 polypeptide, wherein the R5.24 receptor is encoded by a nucleotide sequence that hybridizes under highly stringent hybridization conditions to a nucleotide sequence encoding an amino acid sequence of SEQ ID NO:2, 4, or 6; and
(ii) determining the functional effect of the compound upon the receptor, thereby identifying a compound that modulates signal transduction by the receptor.

19. An isolated magnesium receptor comprising a R5.24 polypeptide, wherein the R5.24 polypeptide is encoded by a nucleotide sequence that hybridizes under highly stringent hybridization conditions to a nucleotide sequence encoding an amino acid sequence of SEQ ID NO:2, 4, or 6.

20. The isolated receptor of claim 19, wherein the R5.24 polypeptide has an amino acid sequence of SEQ ID NO:2, 4, or 6.

21. The isolated receptor of claim 19, wherein the R5.24 polypeptide is encoded by a nucleotide sequence of SEQ ID NO:1, 3, or 5.

22. The isolated receptor of claim 19, wherein the R5.24 polypeptide is recombinant.

23. A host cell comprising the isolated receptor of claim 19.

24. The isolated receptor of claim 19, wherein the receptor binds to magnesium and manganese ligands.

25. The isolated receptor of claim 19, wherein the receptor has G protein coupled receptor activity.

26. An isolated nucleic acid encoding the receptor of claim 19.

27. The nucleic acid of claim 26, wherein the nucleic acid encodes an amino acid sequence of SEQ ID NO:2, 4, or 6.

28. The nucleic acid of claim 26, wherein the nucleic acid has a nucleotide sequence of SEQ ID NO:1, 3, or 5.

29. An expression vector comprising the nucleic acid of claim 26.

30. A host cell comprising the expression vector of claim 29.


SEQUENCE LISTING (7 sequences)

SEQ ID NO: 1 - DNA, 2781 bp, Homo sapiens
  human magnesium/manganese sensing G protein coupled receptor (GPCR) R5.24, Mg receptor, Mn receptor
atggcattcttaattatactaattacctgctttgtgattattcttgctacttcacagccttgccagacccctgatgactttgtggctgccacttctccgggacatatcataattggaggtttgtttgctattcatgaaaaaatgttgtcctcagaagactctcccagacgaccacaaatccaggagtgtgttggctttgaaatatcagtttttcttcaaactcttgccatgatacacagcattgagatgatcaacaattcaacactcttacctggagtcaaactggggtatgaaatctatgacacttgtacagaagtcacagtggcaatggcagccactctgaggtttctttctaaattcaactgctccagagaaactgtggagtttaagtgtgactattccagctacatgccaagagttaaggctgtcataggttctgggtactcagaaataactatggctgtctccaggatgttgaatttacagctcatgccacaggtgggttatgaatcaactgcagaaatcctgagtgacaaaattcgctttccttcatttttacggactgtgcccagtgacttccatcaaattaaagcaatggctcacctgattcagaaatctggttggaactggattggcatcataaccacagatgatgactatggacgattggctcttaacacttttataattcaggctgaagcaaataacgtgtgcatagccttcaaagaggttcttccagcctttctttcagataataccattgaagtcagaatcaatcggacactgaagaaaatcattttagaagcccaggttaatgtcattgtggtatttctgaggcaattccatgtttttgatctcttcaataaagccattgaaatgaatataaataagatgtggattgctagtgataattggtcaactgccaccaagattaccaccattcctaatgttaaaaagattggcaaagttgtagggtttgcctttagaagagggaatatatcctctttccattcctttcttcaaaatctgcacttgcttcccagtgacagtcacaaactcttacatgaatatgccatgcatttatctgcctgcgcatatgtcaaggacactgatttgagtcaatgcatattcaatcattctcaaaggactttggcctacaaggctaacaaggctatagaaaggaacttcgtcatgagaaatgacttcctctgggactatgctgagccaggactcattcatagtattcagcttgcagtgtttgcccttggttatgccattcgggatctgtgtcaagctcgtgactgtcagaaccccaacgcctttcaaccatgggagttacttggtgtgctaaaaaatgtgacattcactgatggatggaattcatttcattttgatgctcacggggatttaaatactggatatgatgttgtgctctggaaggagatcaatggacacatgactgtcactaagatggcagaatatgacctacagaatgatgtcttcatcatcccagatcaggaaacaaaaaatgagttcaggaatcttaagcaaattcaatctaaatgctccaaggaatgcagtcctgggcaaatgaagaaaactacaagaagtcaacacatctgttgctatgaatgtcagaactgtcctgaaaatcattacactaatcagacagatatgcctcactgccttttatgcaacaacaaaactcactgggcccctgttaggagcactatgtgctttgaaaaggaagtggaatatctcaactggaatgactccttggccatcctactcctgattctctccctactgggaatcatatttgttctggttgttggcataatatttacaagaaacctgaacacacctgttgtgaaatcatccgggggattaagagtctgctatgtgatccttctctgtcatttcctcaattttgccagcacgagctttttcattggagaaccacaagacttcacatgtaaaaccaggcagacaatgtttggagtgagctttactctttgcatctcctgcattttgacgaagtctctgaaaattttgctagccttcagctttgatcccaaattacagaaatttctgaagtgcctctatagaccgatccttattatcttcacttgcacgggcatccaggttgtcatttgcacactctggctaatctttgcagcacctactgtagaggtgaatgtctccttgcccagagtcatcatcctggagtgtgaggagggatccatacttgcatttggcaccatgctgggctacattgccatcctggccttcatttgcttcatatttgctttcaaaggcaaatatgagaattacaatgaagccaaattcattacatttggcatgctcatttacttcatagcttggatcacattcatccctatctatgctaccacatttggcaaatatgtaccagctgtggagattattgtcatattaatatctaactatggaatcctgtattgcacattcatccccaaatgctatgttattatttgtaagcaagagattaacacaaagtctgcctttctcaagatgatctacagttattcttcccatagtgtgagcagcattgccctgagtcctgcttcactggactccatgagcggcaatgtcacaatgaccaatcccagctctagtggcaagtctgcaacctggcagaaaagcaaagatcttcaggcacaagcatttgcacacatatgcagggaaaatgccacaagtgtatctaaaactttgcctcgaaaaagaatgtcaagtatatga

SEQ ID NO: 2 - protein, 926 aa, Homo sapiens
  human magnesium/manganese sensing G protein coupled receptor (GPCR) R5.24, Mg receptor, Mn receptor
MAFLIILITCFVIILATSQPCQTPDDFVAATSPGHIIIGGLFAIHEKMLSSEDSPRRPQIQECVGFEISVFLQTLAMIHSIEMINNSTLLPGVKLGYEIYDTCTEVTVAMAATLRFLSKFNCSRETVEFKCDYSSYMPRVKAVIGSGYSEITMAVSRMLNLQLMPQVGYESTAEILSDKIRFPSFLRTVPSDFHQIKAMAHLIQKSGWNWIGIITTDDDYGRLALNTFIIQAEANNVCIAFKEVLPAFLSDNTIEVRINRTLKKIILEAQVNVIVVFLRQFHVFDLFNKAIEMNINKMWIASDNWSTATKITTIPNVKKIGKVVGFAFRRGNISSFHSFLQNLHLLPSDSHKLLHEYAMHLSACAYVKDTDLSQCIFNHSQRTLAYKANKAIERNFVMRNDFLWDYAEPGLIHSIQLAVFALGYAIRDLCQARDCQNPNAFQPWELLGVLKNVTFTDGWNSFHFDAHGDLNTGYDVVLWKEINGHMTVTKMAEYDLQNDVFIIPDQETKNEFRNLKQIQSKCSKECSPGQMKKTTRSQHICCYECQNCPENHYTNQTDMPHCLLCNNKTHWAPVRSTMCFEKEVEYLNWNDSLAILLLILSLLGIIFVLVVGIIFTRNLNTPVVKSSGGLRVCYVILLCHFLNFASTSFFIGEPQDFTCKTRQTMFGVSFTLCISCILTKSLKILLAFSFDPKLQKFLKCLYRPILIIFTCTGIQVVICTLWLIFAAPTVEVNVSLPRVIILECEEGSILAFGTMLGYIAILAFICFIFAFKGKYENYNEAKFITFGMLIYFIAWITFIPIYATTFGKYVPAVEIIVILISNYGILYCTFIPKCYVIICKQEINTKSAFLKMIYSYSSHSVSSIALSPASLDSMSGNVTMTNPSSSGKSATWQKSKDLQAQAFAHICRENATSVSKTLPRKRMSSI

SEQ ID NO: 3 - DNA, 2787 bp, Mus sp.
  mouse magnesium/manganese sensing G protein coupled receptor (GPCR) R5.24, Mg receptor, Mn receptor
atggccctattgattacagtagttacttgcttcatgattattcttgatacctcccagtcttgtcataccccagatgacttcgtggctatcacttctcctggacatatcatgattggtggcttgtttgccattcatgaaaaaatgttgtcctcagatgaccatcccagacgaccacaaatccagaaatgtgctggctttgaaatatcagtgtttcttcaaactctggctatgatacacagcattgagatgatcaataattcaacactgttatccggagtcaagctgggatatgaaatctatgacacttgtaccgaagtcacagcagcaatggctgccactctgaggttcctctcaaaattcaactgctctagagaaactgtggtctttcagtgtgactattccagctacatgccaagggtcaaggctgtcataggtgctggctactctgaaacatccatcgcggtctcaaggatgctgaacttacagctcatgccacaggtgagttatgaatccactgcagaaatcctgagtgacaagatccgctttccttcatttttacggactgtgcctagtgacttctaccaaactaaagcaatggcccacctgatccgacaatcgggatggaactggattggtgccataacaaccgatgatgattatggaagactagctctcaacacatttgcaatacaggctgctgaaaacaatgtgtgcattgccttcaaagaggttctgccagccttcctctcagataataccattgaagtgagaattaaccagactctggagaagatcattgcagaagcccaggttaatgtcattgtggtgtttctgagaaaattccatgttttcaatctcttcacgaaagccattgaaaggaaaataagtaagatctggattgctagtgataattggtcaaccgctaccaagattatcaccatccctaatgttaagaagcttggcaaagtggtggggtttgcctttaggagaggaaatacgtcttctttccattcttttcttcaaactctgcatatgtaccccaatgacaataacaaacccctacatgaatttgccatgctcgtttctgcctgtaaatacatcaaagacggtgatttgagccaatgcatatccaactattctcaggcaactttgacctatgacactaccaagaccattgaaaaccacttattcaagagaaatgatttcctctggcattatactgagccgggactcatttatagcattcagcttgcagtgttcgcccttggtcatgccatccgggatctgtgccaagctcgagactgcaagaaacccaacgcctttcagccatgggagctacttgccgtgctgaaaaacgtgacattcactgatggaaggaattcatttcactttgatgcccacggggatttaaatactggttatgacgtggtgctctggaaggagaccaatggcctcatgactgtcacaaagatggcagaatatgacctgcagcgtgacgtcttcatcaccacaaaccaagaaacaaaacatgaattcaggaaacttaagcaaattctatctaaatgctccaaggaatgcagccctggtcaaatgaagaaagccacaggaagccaacacagctgttgctatgaatgtgtgagctgcccagaaaaccactacagtaatgagacagatatggatcactgccttttatgcaacaatgaaacccactgggccccagtaaggagcactacctgctttgaaaaggaagtggagtatctggactgggatgactccttggctctcctcctcattgccctctccctacttggaatcgcctttgttctggccattggcataatatttacaagaaacctgaagacacctgtcgtgaaatcatctggaggattagtggtctgctacgtgatgctcatctgccacgcccttaactttgccagcacaggcttttttattggagaaccacaagactttgcgtgcaagaccaggcagaccttatttggtgtgagctttactctctgtgtctcctgtattttgaccaagtccctgaaaattttgctagctttcagttttgaccccaagctgacaatgttcctgaagtgcctttataggcctgttcccattgttcttacctgcacgggcattcaagtggtcatttgcactctttggctggtgctggcagcacctagtgtggaagagaatatctccttgcctagagtcattatcctggaatgtgaggaggggtcagcactggcatttggtaccatgctgggctacatcacagttctggccttcatttgctttgtatttgcattcaaggggaggaaacttcctgagaattacaacgaagccaagttcctgacctttgggatgctgatttacttcatagcttggatcacattcatccctgtctatactaccacattcggcaagtatttgcccgccgtggagattatagtcattctgatatccaattatgggattctctgctgtatattcttccccaagtgctacattattctttgcaagcagaagactaacaccaaatcagccttcctccagatggtttacaactactctgctcacagtgtggacagccttgccttgagtcatgtttccctggactccaccagctacgatactgcaacaaccaatcagagccctggtaacaagatgacagcctgtcagaacgacaaccatcttcctgcacaagtgctcccacacacaggcacggcaaagactatcaaagcgtctaaaactttgcgtcagaaaagaagttcaagtatatga

SEQ ID NO: 4 - protein, 928 aa, Mus sp.
  mouse magnesium/manganese sensing G protein coupled receptor (GPCR) R5.24, Mg receptor, Mn receptor
MALLITVVTCFMIILDTSQSCHTPDDFVAITSPGHIMIGGLFAIHEKMLSSDDHPRRPQIQKCAGFEISVFLQTLAMIHSIEMINNSTLLSGVKLGYEIYDTCTEVTAAMAATLRFLSKFNCSRETVVFQCDYSSYMPRVKAVIGAGYSETSIAVSRMLNLQLMPQVSYESTAEILSDKIRFPSFLRTVPSDFYQTKAMAHLIRQSGWNWIGAITTDDDYGRLALNTFAIQAAENNVCIAFKEVLPAFLSDNTIEVRINQTLEKIIAEAQVNVIVVFLRKFHVFNLFTKAIERKISKIWIASDNWSTATKIITIPNVKKLGKVVGFAFRRGNTSSFHSFLQTLHMYPNDNNKPLHEFAMLVSACKYIKDGDLSQCISNYSQATLTYDTTKTIENHLFKRNDFLWHYTEPGLIYSIQLAVFALGHAIRDLCQARDCKKPNAFQPWELLAVLKNVTFTDGRNSFHFDAHGDLNTGYDVVLWKETNGLMTVTKMAEYDLQRDVFITTNQETKHEFRKLKQILSKCSKECSPGQMKKATGSQHSCCYECVSCPENHYSNETDMDHCLLCNNETHWAPVRSTTCFEKEVEYLDWDDSLALLLIALSLLGIAFVLAIGIIFTRNLKTPVVKSSGGLVVCYVMLICHALNFASTGFFIGEPQDFACKTRQTLFGVSFTLCVSCILTKSLKILLAFSFDPKLTMFLKCLYRPVPIVLTCTGIQVVICTLWLVLAAPSVEENISLPRVIILECEEGSALAFGTMLGYITVLAFICFVFAFKGRKLPENYNEAKFLTFGMLIYFIAWITFIPVYTTTFGKYLPAVEIIVILISNYGILCCIFFPKCYIILCKQKTNTKSAFLQMVYNYSAHSVDSLALSHVSLDSTSYDTATTNQSPGNKMTACQNDNHLPAQVLPHTGTAKTIKASKTLRQKRSSSI

SEQ ID NO: 5 - DNA, 2787 bp, Rattus sp.
  rat magnesium/manganese sensing G protein coupled receptor (GPCR) R5.24, Mg receptor, Mn receptor
atggccctatcatttgtatttattacttgcttcatgattcttcttgatacctcccagtcttgtcataccccagatgacttcgtggctatcacttctcctggacatatcatgattggtggtttgtttgccattcacgaaaaaatgttgtcctcagatgaccatcccaggcaaccacaaatccagaagtgtgttggttttgaaatatcagtgtttcttcaaactctggctatgatacacagcattgagatgatcaataactcaagcctgttgtccggagttaagctggggtatgaaatctatgacacttgtactgaagtcacagcggcaatggctgccaccctgaggttcctctctaaattcaactgctctagagaaaccgtgatctttcaatgtgactattccagctacgtgccaagggtcaaggccatcataggtgctggctactctgaaatatccatggcggtctcgaggatgctgaatttacagctcatgccacaggtgagttatgaatccactgcagaaatcctgagtgacaaaatccgctttccttcgtttttacgaaccgtgcccagtgacttctaccaaactaaagcaatggcccacctgatccgacaatccggatggaactgggttggtgccataacaaccgacgatgattatggaagactagcgctcaacacatttgcaatccaggctgctgaaaacaatgtgtgcatcgccttcaaagaggttctgccagccttcctctcagataataccattgaagtgagaatcaaccagactctggagaaaatcattgccgaagcccaggtcaatgtcatcgtggtgtttcttaggaaattccacgtcttcaatctcttcaacaaagccatcgagaggaaaataagtaagatctggattgctagtgataactggtcaactgctgccaagattatcaccattcccaatgttaagaagcttggcaaagtagtgggctttacttttaggagaggaaatatgtcttctttccattcttttcttcaaactctgcatatgtaccccagtgacaataacaaacccctacatgagtttgccatgcttttttctgcctgtaaacacatcaaagatggtgatttgagccaatgcatttccaactattctcaggcaacttggacctatgacactaccaagaccattgagacccatttattcaagagaaatgacttcctttggcattatactgagccaggactcattcacagcattcagcttgctgtgcttgccttgggccatgccatccgggatctgtgccaagatcgagactgccagaaacccaacgcctttcagccatgggagctacttgcggtgctgaaaaacgtgacattcactgacggaaaaaactcatttcattttgatgcccatggggatttaaatactggttacgaggtggtgctctggaaagagactaacggcctcatgactgtcacgaagatggcagaatatgacctgcagcatgacgtcttcatcaccacaaaccaagaaacaaagcatgaattcaggaaacttaagcaaattctatctaaatgctccaaagaatgcattcctggtcaaatgaagaaagccacaggaagccaacacagctgttgctatgaatgtgtgaactgcccagaaaaccactacagtaatgagacagacatggatcactgcctcgtatgcaacaacgaaacccactgggccccagtaaggagcacgatgtgcttcgagaaggaagtggagtatcttgactgggatgactccttggctctcctcctcattgccctctccctacttggaattgcctttgttctggccgttggcataatatttacaagaaacctgaagacacctgtggtgaaatcatctgggggattagtggtctgttacgtgatgctcgcctgccatgccctcaactttgccagcacaggcttttttattggagaaccacaggacttcacgtgcaagaccaggcagaccctgtttggcgtgagctttactctgtgtgtctcctgtattttgacgaagtccctgaaaattttgctagccttcagcttcgaccccacgctgaagacattcctgaagtgtctctataggccagtccccattgtccttacctgcacgggcattcaagtggtcatttgcacgctttggctggtcctggcagcacctactgtggaagagaacacctccttgcctagagtcattatcctagaatgtgaggagggatctgcactggcgtttggcaccatgctgggctacattgcagttctggccttcatttgttttgtatttgcattcaaggggaggaagcttcctgagaattacaacgaagctaagttcctgacctttgggatgctgatttacttcatagcttggatcacattcatcccagtctatgctaccactttcggcaagtatttgcccgctgtggagatcatagtcattctgatatccaactatggtatcctctgctgtacattcttccccaagtgctacattattctttgcaagcaaaagactaacactaagtcagtcttcctccagatggtttacaattactctgctcacagcgtggacagccttgccttgagtcacgtatccctggactccgccagccacagtactgcaacaaccaacccgaggcctggtaacaagactgcagcctgtcagaattacaaacatcttcctgtacaagtgcttgctcacacaggcatggaaaagactatgcacgcgtctaaaactttgcatcagaaaagaagttcaagtatatga

SEQ ID NO: 6 - protein, 928 aa, Rattus sp.
  rat magnesium/manganese sensing G protein coupled receptor (GPCR) R5.24, Mg receptor, Mn receptor
MALSFVFITCFMILLDTSQSCHTPDDFVAITSPGHIMIGGLFAIHEKMLSSDDHPRQPQIQKCVGFEISVFLQTLAMIHSIEMINNSSLLSGVKLGYEIYDTCTEVTAAMAATLRFLSKFNCSRETVIFQCDYSSYVPRVKAIIGAGYSEISMAVSRMLNLQLMPQVSYESTAEILSDKIRFPSFLRTVPSDFYQTKAMAHLIRQSGWNWVGAITTDDDYGRLALNTFAIQAAENNVCIAFKEVLPAFLSDNTIEVRINQTLEKIIAEAQVNVIVVFLRKFHVFNLFNKAIERKISKIWIASDNWSTAAKIITIPNVKKLGKVVGFTFRRGNMSSFHSFLQTLHMYPSDNNKPLHEFAMLFSACKHIKDGDLSQCISNYSQATWTYDTTKTIETHLFKRNDFLWHYTEPGLIHSIQLAVLALGHAIRDLCQDRDCQKPNAFQPWELLAVLKNVTFTDGKNSFHFDAHGDLNTGYEVVLWKETNGLMTVTKMAEYDLQHDVFITTNQETKHEFRKLKQILSKCSKECIPGQMKKATGSQHSCCYECVNCPENHYSNETDMDHCLVCNNETHWAPVRSTMCFEKEVEYLDWDDSLALLLIALSLLGIAFVLAVGIIFTRNLKTPVVKSSGGLVVCYVMLACHALNFASTGFFIGEPQDFTCKTRQTLFGVSFTLCVSCILTKSLKILLAFSFDPTLKTFLKCLYRPVPIVLTCTGIQVVICTLWLVLAAPTVEENTSLPRVIILECEEGSALAFGTMLGYIAVLAFICFVFAFKGRKLPENYNEAKFLTFGMLIYFIAWITFIPVYATTFGKYLPAVEIIVILISNYGILCCTFFPKCYIILCKQKTNTKSVFLQMVYNYSAHSVDSLALSHVSLDSASHSTATTNPRPGNKTAACQNYKHLPVQVLAHTGMEKTMHASKTLHQKRSSSI

SEQ ID NO: 7 - protein, 200 aa, Artificial Sequence
  Description of Artificial Sequencepoly Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG